(19) United States
(12) Patent Application Publication
(10) Pub. No.: US 2025/0306010 A1
(43) Pub. Date: Oct. 2, 2025

(54) BIOSENSOR FOR MALE INFERTILITY

(71) Applicant: Spermosens AB, Lund (SE)

(72) Inventors: Kushagr Punyani, Lund (SE); Sudha Srivastava, Lund (SE); Mohamad Takwa, Lund (SE)

(21) Appl. No.: 19/092,356

(22) Filed: Mar. 27, 2025

Related U.S. Application Data
(63) Continuation of application No. 17/260,609, filed on Jan. 15, 2021, now abandoned.

(30) Foreign Application Priority Data
Aug. 24, 2018 (EP) 18190720.5

(51) Int. Cl. G01N 33/50; G01N 27/327; G01N 33/543; G01N 33/68

(52) U.S. Cl. CPC G01N 33/5044; G01N 27/3277; G01N 33/5438; G01N 33/689

(57) ABSTRACT

The present invention relates to a biosensor and applications thereof for the quantification of sperm function. Methods and tools for diagnosis of male infertility are also disclosed herein.

### CROSS REFERENCE TO RELATED APPLICATIONS

This application depends from and claims priority to U.S. patent application Ser. No. 17/260,609 filed Jan. 15, 2021, and which is a U.S. National Stage under 35 U.S.C. § 371 of international application number PCT/EP2019/072574 filed Aug. 23, 2019, and which claims priority to European Patent Application Number 18190720.5 filed Aug. 24, 2018, the entire contents of each of which are incorporated by reference herein.

### SEQUENCE LISTING

The instant application contains a Sequence Listing which has been submitted in.xml format via USPTO Patent Center and is hereby incorporated by reference in its entirety. Said .xml copy, created on Mar. 27, 2025, is named “SPER0001NA1 Sequence Listing.xml” and is 7,660 bytes in size.

### TECHNICAL FIELD

The present invention relates to a biosensor and applications thereof for the quantification of sperm function and evaluation of male fertility.

### BACKGROUND

Around 15% of the global population is affected by infertility, wherein male infertility is known to contribute to 20-70% of all the cases (Reprod Biol Endocrinol. 2015, 13:37). It has further been estimated that 2.5-12% of all men are infertile. With the declining birth rates, and other male infertility-associated factors in Europe, the situation is alarming. As treatment of male infertility relies on accurate diagnosis, the analysis and detection of the underlying cause is critical. Besides anatomical and endocrine analyses, current trends in laboratory diagnosis of male infertility span sperm and semen characteristics (Mayo Clinic). 30-40% of the cases of male infertility are related to unknown male infertility-associated factors (European Association of Urology, 2015). This is known as idiopathic male infertility, treatment of which calls for exploring detailed functional analyses.

Further, Assisted Reproductive Technology (ART) is a standard treatment option that also encounters this roadblock. In 2015, 1.6% of all infants in U.S. were conceived via ART. Although 231,936 ART cycles were performed in U.S in 2015 alone, they led to 60,778 live births (Centers for Disease Control and Prevention, 2016). In 2017, the European Society of Human Reproduction and Embryology identified failed fertilization rate (FFR) of the oocyte after regular In Vitro Fertilization (IVF) as a Key Performance Indicator for ART labs, with a very low competence level of 5% (Human Reprod Open. 2017, 2). It is important to note that while sperm morphology cannot be correlated to IVF success/failure, failure of fertilization is attributed to issues with sperm function (Human Reprod. 2000, 15(3): 702; Fertil Steril. 2003 January;79(1):74). Thus there is a need for development of a robust method for diagnosing the fertilization competence of sperm cells in order to predict the viability of IVF, and better select the appropriate ART method.

Before ART, typically male-infertility-associated factors are probed and include sperm quality analysis, sperm counts, concentration, morphology and motility, identification of atypical cell types in semen, and presence of autoimmune antibodies. Additional analyses may involve studying the interaction of sperm cells with cervical mucus, acrosomal reaction, biochemical assays for accessory sex organ function, and estimation of reactive oxygen species and DNA damage (World Health Organization, 2010). In cases of idiopathic male infertility and failure of fertilization in IVF, these values do not provide much information on the underlying cause of infertility. Resorting to intracytoplasmic sperm injection (ICSI) is most often the preferred methodology, but the unnecessary step of IVF leads to wastage of healthy oocytes and financial load.

While the morphological and kinetic properties of sperm cells are crucial for fertilization in vivo or in IVF, the ultimate step in fertilization is the fusion of sperm cells with the ovum. Research methods like Hemizona assay, Human sperm-oocyte interaction test and Human zona pellucida binding test can mimic parts of this step. However, these methods cannot be commercialized due to their reliance on human oocytes, or parts thereof, which are not readily available. Zona-free hamster oocyte penetration test was developed for the purpose of using hamster oocytes instead of human oocytes. While the test negates the requirement for human oocytes, it has a poor predictive value for the success of fertilization in IVF treatment at any insemination concentration, and the usage of this test is therefore significantly limited.

The primary binding between the two gametes is mediated by the extracellular layer of Zona pellucida (ZP) glycoproteins surrounding the ova (Cell. 2017, 169(7):1315; Reprod Biomed Online. 2003, 7(6):641). This interaction is responsible for triggering the acrosomal reaction in sperm cells. Further, sperm cells that have not commenced acrosomal reaction prior to encountering ZP are not able to fertilize the ova. Next, the hydrolases released from the acrosome need to digest the ZP, thereby permitting the sperm to make its way to the ova membrane.

The crucial step in this binding was discovered in 2014. The sperm surface antigen IZUMO1 binds to the female counterpart JUNO protein, formerly known as Folate receptor 4 (Nature. 508:483-487; Nature. 2016, 534(7608):566). This biochemical event has been discovered to be essential for fusion of the two gametes. Any biochemical mismatch may lead to failure of fertilization.

Hence, being able to quantify the amount of sperm cells in a semen sample capable of binding to the JUNO protein is important in the evaluation of fertility.

A biosensor is a sensor that utilizes the molecule-identifying function of a biological material, e.g. a microorganism, enzyme, antibody, DNA, and RNA, and applies such a biological material as a molecule-identifying element. In other words, the biosensor utilizes the reaction occurring when an immobilized biological material identifies a target substrate, oxygen consumed by breathing of microorganism, enzyme reaction, luminescence, and the like. Among biosensors, practical use of enzyme sensors is developing. For example, enzyme sensors for glucose, lactic acid, uric acid, and amino acids find applications in medical instrumentation and food processing industry.

Different techniques may be used to follow the interaction between for example a protein bound to an electrode and the target species, such as the sperm. One of such techniques relies on Surface Plasmon Resonance (SPR). In SPR, one molecular partner, such as a protein, is immobilized on a metal (the chip). Light excites surface plasmons in the metal; when the binding partner binds to the immobilized molecule, this causes a detectable change in the surface plasmon signal. Another of such techniques relies on electrochemical transduction in which the content of a biological sample is analyzed by the direct conversion of a biological event to an electronic signal. The most common techniques in electrochemical biosensing comprise cyclic voltammetry, chronoamperometry, chronopotentiometry, impedance spectroscopy, and field-effect transistor based methods along with nanowire or magnetic nanoparticle-based biosensing.

### SUMMARY

The present inventors utilize the biochemical reaction between the JUNO protein and the sperm surface antigen IZUMO1 in order to discern the fertilization potential of sperm cells. Besides revealing the underlying cause of infertility, this will especially be useful for selection of suitable ART techniques, i.e. a choice between regular IVF and ICSI, while minimizing the wastage of ova. The present inventors further propose development and clinical validation of an electrochemical and/or optical sensing platform(s) that probes the fertilization potential of sperm cells in order to diagnose male infertility. The existing commercial methods to diagnose male infertility and check the sperm quality look into physical aspects of the sperm cells, while overlooking the biochemical interaction that is necessary for the fertilization event. The proposed method has advantages over the existing diagnostic methods, as it is the first bioinspired assay for male fertility analysis that exploits bioreceptors of sperm cells, and it mimics crucial steps of fertilization by the sperm cells thereby giving a direct insight into their fertilization potential. Previously, sperm-oocyte interaction tests have been developed, but they all either require using human oocytes and zona pellucidae, which are not easily available, or they are unreliable due to having a low correlation between the test results and the various semen parameters. The present invention overcomes the problem of oocytes availability by creating conditions that mimic the oocyte and in particular by using one or more of the crucial protein receptors involved in sperm-oocyte fusion.

It is an aspect of the present disclosure to provide a biosensor for quantification of sperm function, the biosensor comprising a substrate and a JUNO protein or a fragment thereof, wherein the JUNO protein or fragment thereof is immobilized on the substrate.

It is a further aspect of the present disclosure to provide a biosensor for detection of sperm function, the biosensor comprising a substrate and a JUNO protein or a fragment thereof, wherein the JUNO protein or fragment thereof is immobilized on the substrate.

It is also an aspect of the present disclosure to provide a method for detecting and/or quantifying sperm function, wherein the method comprises the steps of:
a. Providing a semen sample from a subject, wherein said semen sample comprises one or more sperm cells,
b. Contacting the semen sample with the biosensor according to any one of the preceding claims,
c. Determining binding of the sperm cells to a protein immobilized on the sensor,
   thereby detecting and/or quantifying the sperm function of said sample.

It is also an aspect of the present disclosure to provide a method for diagnosis of male infertility, wherein the method comprises the steps of:
a. Providing a semen sample from a subject,
b. Contacting the semen sample with the biosensor according to the present disclosure,
c. Quantifying the sperm function of said sample according to the method of the present disclosure,
d. Using the sperm function to diagnose if the subject is infertile.

It is also an aspect of the present disclosure to provide a method for diagnosis of male infertility, wherein the method comprises the steps of:
a. Providing a sperm sample from a subject,
b. Contacting the sperm sample with the biosensor according to the present disclosure,
c. Quantifying the sperm function of said sample according to the method of the present disclosure,
d. Using the sperm function to diagnose if the subject is infertile.

Another aspect of the present disclosure is the provision of a method for manufacturing a biosensor comprising a JUNO protein, the method comprising:
a. Providing a substrate,
b. providing the JUNO protein,
c. immobilizing the JUNO protein on the substrate,
   thereby manufacturing a biosensor comprising the JUNO protein.

It is a further aspect of the present disclosure to provide a method of selecting sperm, said method comprising:
a. Providing a semen sample from a subject, wherein said semen sample comprises one or more sperm cells,
b. Contacting the semen sample with the biosensor according to the present disclosure,
c. Visualizing a sperm bound to the biosensor by microscopy,
   thereby selecting said sperm.

It is a further aspect of the present disclosure to provide a method of selecting sperm, said method comprising:
a. Providing a sperm sample from a subject,
b. Contacting the sperm sample with the biosensor according to the present disclosure,
c. Visualizing a sperm bound to the biosensor by microscopy,
   thereby selecting said sperm.

It is also an aspect of the present disclosure to provide a hand-held device for detection and/or quantification of sperm function, the device comprising:
a. An inlet for a sample;
b. A biosensor comprising a JUNO protein or a fragment thereof, wherein the JUNO protein is immobilized on the biosensor, and wherein the inlet is configured to place the sample in contact with the sensor;
c. A detector configured to receive a signal from the sensor and transform it into a format readable by a user;
d. Optionally, means for separating cellular components from the sample.

### DESCRIPTION OF THE DRAWINGS

FIG. 1: Cyclic voltammetric response of a) Electrode-gold nanoparticles-BSAblocking after addition of buffer only (solid line) and after addition of semen sample diluted 1.67×10⁻²X in buffer (dotted line) b) Electrode-gold nanoparticles-JUNO-BSAblocking (dash dot dot line) and c) Electrode-gold nanoparticles-ZP3-BSAblocking response (dashed line) after addition of semen sample diluted 1.67×10⁻²X in buffer, with the dilution factor given by the initial volume/final volume.

FIG. 2A to FIG. 2F: Cyclic voltammetric response of Electrode-gold nanoparticles-ZP3-BSAblocking after addition of three different dilutions (1.56×10⁻⁵X (dotted line), 1.67×10⁻²X (dash dot dot line) and 2.5×10⁻²X (dashed line)) of 6 different semen samples (FIG. 2A-FIG. 2F), while solid line shows electrode response with buffer only (no semen sample).

FIG. 3A to FIG. 3F: Cyclic voltammetric response of Electrode-gold nanoparticles-JUNO-BSAblocking after addition of three different dilutions (1.56×10⁻⁵X (dotted line), 1.67×10⁻²X (dash dot dot line) and 2.5×10⁻²X (dashed line)) of semen samples S1, S2, S3, S4, S5 and S6 (FIG. 3A-FIG. 3F, respectively), while solid line shows electrode response with buffer only (no semen sample).

### DETAILED DESCRIPTION

Disclosed herein is a biosensor and applications thereof for the quantification of sperm function to evaluate male infertility. Further, the present disclosure relates to methods for diagnosis of infertility in a subject comprising determining the sperm function of sperm cells in a sample obtained from said subject.

### Sperm Function

The main function of sperm is to reach the ovum to induce fertilization by fusing with it to deliver two sub-cellular structures: (i) the male pronucleus that contains the genetic material and (ii) the centrioles that are structures that help organize the microtubule cytoskeleton. Hence, sperm function can be understood as the sperm's ability to reach the ovum and induce fertilization.

The primary binding between the two gametes is mediated by the extracellular layer of Zona pellucida (ZP) glycoproteins surrounding the ova (Cell. 2017, 169(7):1315; Reprod Biomed Online. 2003, 7(6):641). This interaction is responsible for triggering the acrosomal reaction in sperm cells. Further, sperm cells that have not commenced acrosomal reaction prior to encountering ZP are not able to fertilize the ova. Next, the hydrolases released from the acrosome need to digest the ZP, thereby permitting the sperm to make its way to the ova membrane.

The crucial step in this binding was discovered in 2014. The sperm surface antigen IZUMO1 binds to the female counterpart JUNO protein, formerly known as Folate receptor 4 (Nature. 508: 483-487; Nature. 2016, 534(7608):566). This biochemical event has been discovered to be essential for fusion of the two gametes.

The present disclosure is directed to a biosensor for quantification of sperm function, the biosensor comprising a substrate and a JUNO protein or a fragment thereof, wherein the JUNO protein or fragment thereof is immobilized on the substrate. In fact, probing sperm function through binding of sperm cells to a biosensor comprising the JUNO protein may be a viable strategy, and may ultimately allow diagnosis of male infertility, that overcomes the need for human or animal oocytes or parts thereof.

In some embodiments, a biosensor according to the present disclosure is provided, wherein the sperm function is determined from the binding of at least a portion of sperm to a protein, or a fragment thereof, which has been immobilized on the sensor, wherein said protein(s) is JUNO protein, ZP1, ZP2, ZP3 and/or an anti-IZUMO antibody, or fragments thereof. In some embodiments, the at least a portion of sperm comprises an IZUMO1 surface antigen.

It is an aspect of the disclosure to provide a method for detecting and/or quantifying sperm function, wherein the method comprises the steps of:
a. Providing a semen sample from a subject, wherein said semen sample comprises one or more sperm cells,
b. Contacting the semen sample with the biosensor according to any one of the preceding claims,
c. determining binding of the sperm cells to a protein immobilized on the sensor,
   thereby detecting and/or quantifying the sperm function of said sample.

It is a further aspect of the present disclosure to provide a method for diagnosis of male infertility, wherein the method comprises the steps of:
a. Providing a semen sample from a subject,
b. Contacting the semen sample with the biosensor according to the present disclosure,
c. Quantifying the sperm function of said sample according to the method disclosed herein,
d. Using the sperm function to diagnose if the subject is infertile.

It is another aspect of the present disclosure to provide a method for diagnosis of male infertility, wherein the method comprises the steps of:
a. Providing a sperm sample from a subject,
b. Contacting the sperm sample with the biosensor according to the present disclosure,
c. Quantifying the sperm function of said sample according to the method disclosed herein,
d. Using the sperm function to diagnose if the subject is infertile.

The term “sperm function” as used herein refers to the sperm health, which is the ability a sperm to capacitate and to fertilize an egg. Sperm function tests are diagnostic or research methods that probe the biochemical or molecular traits of sperm cells. The following reference has several examples of sperm function tests: Talwar and Hayatnagarkar 2015. J Hum Reprod Sci. 8(2): 61-69. The present disclosure relates to sperm function tests which are also referred to as sperm-oocyte interaction tests, wherein the tests, instead of using an oocyte, mimic the conditions of sperm-oocyte interaction by using one or more of the crucial protein receptors involved in sperm-oocyte fusion.

In one embodiment according to the method of the present disclosure, the semen sample comprises, or is suspected of comprising, one or more sperm cells.

In one embodiment according to the method of the present disclosure, the sperm function of said sample is detected by determining binding of the sperm cells to a protein immobilized on the sensor, wherein said protein is selected from the group consisting of JUNO protein, ZP1, ZP2, ZP3 and/or the anti-IZUMO antibody, or fragments thereof, and wherein said binding is detected by microscopic analysis, electrochemical detection and/or surface plasmon resonance.

In one embodiment according to the method of the present disclosure, the sperm function of said sample is quantified by determining binding of the sperm cells to a protein immobilized on the sensor, wherein said protein is selected from the group consisting of JUNO protein, ZP1, ZP2, ZP3 and/or the anti-IZUMO antibody, or fragments thereof, and wherein said binding is detected by microscopic analysis, electrochemical detection and/or surface plasmon resonance.

In one embodiment according to the method of the present disclosure, the sperm function is quantified in step c) by determining the percentage of bound versus unbound sperm cells by microscopic analysis.

In one embodiment according to the method of the present disclosure, the sperm function is quantified in step c) by determining the percentage of bound versus unbound sperm cells by electrochemical detection.

In one embodiment according to the method of the present disclosure, the sperm function is quantified in step c) by determining the percentage of bound versus unbound sperm cells by surface plasmon resonance.

In one embodiment according to the method of the present disclosure, the sperm function is quantified in step c) by determining the acrosomal status of the sperm cells in the semen sample, and/or in the sperm sample, by microscopic analysis.

In one embodiment according to the method of the present disclosure, the sperm function is quantified in step c) by determining the acrosomal status of the sperm cells in the semen sample, and/or in the sperm sample, by electrochemical detection.

In one embodiment according to the method of the present disclosure, the sperm function is quantified in step c by determining the acrosomal status of the sperm cells in the semen sample, and/or in the sperm sample, by surface plasmon resonance.

In one embodiment according to the method of the present disclosure, said method further comprises comparing the percentage of bound versus unbound sperm cells and/or the acrosomal status of the sperm cells with respective reference values, wherein said reference values may be positive reference values (representing functional sperm) and/or negative reference values (representing non-functional sperm). Said reference values may be obtained by testing control semen samples, and/or control sperm samples. Said reference values may also be obtained from data available in the scientific literature.

In one embodiment according to the method of the present disclosure, the sample is treated prior to step b. For example, the semen sample treatment may comprise liquefaction of the sperm. The semen sample treatment may optionally comprise capacitation. The sperm sample treatment may comprise liquefaction of the sperm. The sperm sample treatment may optionally comprise capacitation.

The biosensor disclosed herein may be used to determine sperm function at various levels.

For example, the disclosed biosensor may be used to determine the capability of the sperm cells in the semen sample, and/or in the sperm sample, to bind to the zona pellucidae proteins ZP1, ZP2 and/or ZP3.

The disclosed biosensor may be used to determine the capability of the sperm cells in the semen sample, and/or in the sperm sample, to undergo acrosomal reaction.

Binding to the zona pellucidae proteins ZP1, ZP2 and/or ZP3 is a necessary step for a sperm cell in order to undergo acrosomal reaction. Hence, sperm cells that are not capable of binding to the zona pellucidae proteins ZP1, ZP2 and/or ZP3 will not undergo acrosomal reaction.

Determining whether sperm cells are capable of binding to the zona pellucidae proteins ZP1, ZP2 and/or ZP3 and undergoing acrosomal reaction is important for establishing which assisted reproduction techniques may be used. In particular, sperm cells that are not capable of binding to the zona pellucidae proteins ZP1, ZP2 and/or ZP3 and/or do not undergo acrosomal reaction, may be suitable for IVF, provided that the zona pellucida coat has been removed from the egg.

The disclosed biosensor may be used to determine the capability of the sperm cells in the semen sample, and/or in the sperm sample, to bind a JUNO protein.

A sperm cell may be able to bind a JUNO protein even though said sperm cell is not capable of binding to the zona pellucidae proteins ZP1, ZP2 and/or ZP3, and/or does not undergo acrosomal reaction. This is because the sperm cell may be induced to capacitate and expose surface antigens necessary for binding to the oocyte's cell membrane.

In some embodiments, the sperm function is determined by the ability of a sperm to bind to the immobilized JUNO protein or fragment thereof. Said binding may for example occur via an IZUMO1 protein or fragment thereof situated on the sperm. Said binding may for example occur via an IZUMO1 protein or fragment thereof expressed by the sperm.

In fact, a sperm that has undergone capacitation may present an IZUMO1 protein or fragment thereof on its surface and said IZUMO1 protein or fragment thereof is capable of binding its egg receptor counterpart, for example a JUNO protein or fragment thereof, or an anti-IZUMO antibody or fragment thereof.

In some embodiments, the sperm function is determined by the binding of an IZUMO1 protein or fragment thereof to the immobilized JUNO protein or fragment thereof.

In a particular embodiment, the method according to the present disclosure further comprises a step of treating said male infertility.

In a particular embodiment of the method according to the present disclosure the treatment comprises administration of a medicament in a therapeutically effective amount and/or by artificial reproductive technology (ART). For example, a subject diagnosed with reduced sperm function may undergo reproduction with the help of ART such as intrauterine insemination (IUI), in vitro fertilization (IVF), or IVF with intracytoplasmatic sperm injection (ICSI).

For example, a subject diagnosed with reduced sperm function, in particular with sperm cells characterized by:
A reduced capability of binding to the zona pellucidae proteins ZP1, ZP2 and/or ZP3,
A reduced capability of undergoing acrosomal reaction;
A normal capability of binding a JUNO protein,
   may undergo reproduction with the help of IVF.

For example, a subject diagnosed with reduced sperm function, in particular with sperm cells characterized by:
a reduced capability of binding to the zona pellucidae proteins ZP1, ZP2 and/or ZP3,
a reduced capability of undergoing acrosomal reaction;
a reduced capability of binding a JUNO protein,
   may undergo reproduction with the help of IVF with ICSI.

The terms “reduced capability” and “normal capability” as used herein are in relation to a reference value, which may be a positive control. The reference value may be obtained by calculating the average values for semen samples of fertile subjects. The reference value may be obtained by calculating the average values for sperm samples of fertile subjects. The reference values may also be obtained from scientific reports. Hence, in order to diagnose a subject with reduced sperm function and/or male infertility, the capability of sperm cells taken from a semen sample, and/or a sperm sample, of said subject of binding to the zona pellucidae proteins ZP1, ZP2 and/or ZP3, and/or of undergoing acrosomal reaction, is compared to at least a reference value, such as a positive control and/or a negative control.

Any treatment for male infertility known to the person skilled in the art may be used.

In some embodiments, the biosensor according to the present disclosure is configured for detection and/or quantification of sperm function.

In one embodiment according to the method of the present disclosure, the sperm function is quantified in step c) by determining the acrosomal status of the sperm cells in the semen sample, and/or in the sperm sample, which results in a translation of the extent of acrosomal reactivity to a measure of sperm function. In a particular embodiment, a fluorophore is added to the sample, followed by a step of analysing said sample by SPR or microscopy to determine the acrosomal status (that is if the sperm cells are “acrosome-reacted”) of the sperm cells. In some embodiments, the fluorophore is added to the sample in the inlet of the biosensor only after having contacted said sample with the substrate. In some embodiments, the fluorophore is added to the sample in the inlet of the biosensor only after the acrosomal reaction has occurred. The presence of a fluorophore may facilitate the step of determining the acrosomal status of the sperm cells. For example fluorescently labelled lectins, such as Pisum sativum (pea agglutinin) or Arachis hypogaea (peanut lectin), or monoclonal antibodies against the acrosome antigen CD46 can be used to assess the acrosomal status of sperm cells. Methods for detecting acrosomal reactions may be found in the WHO laboratory manual for the Examination and processing of human semen, WHO, 5^{th} Edition, 2010, ISBN 978 92 4 154778 9 (see in particular Chapter 4).

In some embodiments according to the method of the present disclosure, the sperm function is quantified in step c) by determining the acrosomal status of the sperm cells in the semen sample, and/or in the sperm sample, which results in a translation of the extent of acrosomal reactivity to a measure of sperm function, wherein the acrosomal reactivity of the semen sample, and/or in the sperm sample, is compared to the average acrosomal reactivity of sperm collected from fertile male individuals, and wherein functional sperm may have acrosomal reactivity equal or higher than the average acrosomal reactivity of sperm collected from fertile male individuals. Data regarding the average acrosomal reactivity of sperm collected from fertile male individuals may be found in the scientific literature and clinical reports.

In some embodiments according to the method of the present disclosure, the sperm function is quantified in step c) by translating the extent of acrosomal reactivity to a measure of sperm function, wherein acrosomal reactivity of 15% or more may be indicative of functional sperm.

In some embodiments according to the method of the present disclosure, the sperm function is quantified in step c) by translating the extent of acrosomal reactivity to a measure of sperm function, wherein acrosomal reactivity of 10% or less may be indicative of non-functional sperm.

In some embodiments according to the method of the present disclosure, the sperm function is quantified in step c) by translating the extent of acrosomal reactivity to a measure of sperm function, wherein acrosomal reactivity of between 10 and 15% may be indicative of abnormal sperm function.

### Acrosome Reaction

During fertilization, a sperm must first fuse with the plasma membrane and then penetrate the female egg in order to fertilize it. Prior to penetrating through the egg's hard shell or extracellular matrix, sperm cells undergo a process known as the acrosome reaction. The acrosome reaction is an exocytotic process that occurs after spermatozoa bind to the zona pellucida and must take place before the spermatozoon can penetrate the oocyte vestments and fuse with the oocyte. The acrosome is a cap-like structure over the anterior half of the sperm's head. As the sperm approaches the zona pellucida of the egg, which is necessary for initiating the acrosome reaction, the membrane surrounding the acrosome fuses with the plasma membrane of the sperm's head, exposing the contents of the acrosome. The contents include surface antigens necessary for binding to the egg's cell membrane, and numerous enzymes, which are responsible for breaking through the egg's tough coating and allowing fertilization to occur. For example, the content of the acrosome may comprise an IZUMO1 protein or fragment thereof.

### The Biosensor

It is an aspect of the present disclosure to provide a biosensor for quantification of sperm function, the biosensor comprising a substrate and a JUNO protein or a fragment thereof, wherein the JUNO protein or fragment thereof is immobilized on the substrate.

It is also an aspect of the present disclosure to provide a biosensor for detection of sperm function, the biosensor comprising a substrate and a JUNO protein or a fragment thereof, wherein the JUNO protein or fragment thereof is immobilized on the substrate.

In one embodiment, the biosensor according to the present disclosure is a sensor. The sensor or biosensor of the present disclosure is configured for detection and/or quantification of sperm function.

The “biosensor”, as used herein is sometimes referred to as a “sensor”. A variety of devices for detecting ligand/receptor interactions are known. The most basic of these are purely chemical/enzymatic assays in which the presence or amount of analyte is detected by measuring or quantitating a detectable reaction product. Ligand/receptor interactions can also be detected and quantitated by radiolabel assays.

Quantitative binding assays of this type involve two separate components: a reaction substrate, e.g., a solid-phase test strip, a dish, a chip or an electrode, and a separate reader or detector device, such as a scintillation counter, spectrophotometer, a microscope, or any other detector known in the art. The substrate is generally unsuited to multiple assays, or to miniaturization, for handling multiple analyte assays from a small amount of body-fluid sample.

In biosensors, by contrast, the assay substrate and detector surface may be integrated into a single device. One general type of biosensor employs an electrode surface in combination with current or impedance measuring elements for detecting a change in current or impedance in response to the presence of a ligand-receptor binding event. Another type of biosensor may employ a chip, for example a glass chip, in combination with an optical detector, for example in combination with surface plasmon resonance. Another type of biosensor may employ a dish in combination with a microscope. A further type of biosensor may employ a microbead in combination with an optical or electrical detector, for example suitable for a latex agglutination test. An even further type of biosensor may employ a polymer substrate, such as a cellulose or nitrocellulose paper, in combination with an optical or electrical detector, for example suitable for a lateral flow test.

The term “dish” as used herein may refer to a vessel or a slide made of glass, ceramic, plastic, cellulose, nitrocellulose or any other material, and can be used as a substrate for microscopic or macroscopic optical detection and selection. Examples include glass slides, microtiter plates, multi-well plates, Petri dishes, watchglasses, etc. The dish may be made of a material that may be modified for example coated with a layer of gold.

The term “microbead” as used herein refers to a particle having a diameter of 1 mm or less. Microbeads may be made of natural or synthetic polymeric materials. Microbeads may be made of a material characterized by having a surface that can be modified, for example they may have a surface that may be conjugated to nanoparticles, such as gold nanoparticles, and/or molecules, such as peptides.

A biosensor refers to a sensor comprising a biological element. Biosensors are practically substitutes of conventional analytical techniques that may be tedious, costly, complex and not appropriate for in situ supervising. A biosensor may be a chemical analytical device unifying a biological element with a transducer. It consolidates a biological element within or in close contact with a transducer which yields a signal proportional to a single analyte that is further conveyed to a detector. In some embodiments, the signal output from the binding of the analyte may be visualized by microscopy. In some embodiments, the signal output from the binding of the analyte may be visualized by an optical detector.

A biosensor embraces three fundamental components that are a bioreceptor (the biological element), a transducer and an electronic circuit. The bioreceptor or biological element is a biomolecule that is embedded with the transducer, like an enzyme, DNA, protein, whole cell, antibodies etc. In the present application, the bioreceptor may be the JUNO protein. In some embodiments of the present disclosure, the biosensor comprises more than one type of bioreceptors, for example a type of bioreceptor may be a JUNO protein or a fragment thereof, and another type of bioreceptor may be selected from a group consisting of ZP1, ZP2, ZP3, and an anti-IZUMO antibody.

Detectors encompassed by the methods of the present disclosure are optical detectors, such as a surface plasmon resonance detector, electrochemical detectors, and measurement circuits. Electronic circuit comprises a signal processing biosensor that converts an electrical signal into a processable signal. In some embodiments, the detector may be a microscope.

Biosensors based on surface plasmon resonance (SPR) effects exploit the shift in SPR surface reflection angle that occurs with perturbations, e.g., binding events, at the SPR interface. Finally, biosensors may also utilize changes in optical properties at a biosensor surface.

Electrochemical biosensors are normally based on enzymatic catalysis of a reaction that produces or consumes electrons (redox enzymes). The sensor substrate usually contains three electrodes; a reference electrode, a working electrode and a counter electrode. The target analyte is involved in the reaction that takes place on the active electrode surface, and the reaction may cause either electron transfer across the double layer (producing a current) or can contribute to the double layer potential (producing a voltage). Either the current can be measured, wherein the rate of flow of electrons is proportional to the analyte concentration at a fixed potential or the potential can be measured at zero current, which gives a logarithmic response. Further, the label-free and direct electrical detection of small peptides and proteins is possible by their intrinsic charges using biofunctionalized ion-sensitive field-effect transistors.

Potentiometric biosensors, in which potential is produced at zero current, gives a logarithmic response with a high dynamic range. Such biosensors are often made by screen printing the electrode patterns on a plastic substrate, coated with a conducting polymer and then some protein (enzyme or antibody) is attached. They have only two electrodes and are extremely sensitive and robust. They enable the detection of analytes at levels previously only achievable by HPLC and LC/MS and without rigorous sample preparation. All biosensors usually involve minimal sample preparation as the biological sensing component is highly selective for the analyte concerned. The signal is produced by electrochemical and physical changes in the conducting polymer layer due to changes occurring at the surface of the sensor. Such changes can be attributed to ionic strength, pH, hydration and redox reactions. Field effect transistors (FET), in which the gate region has been modified with an enzyme or antibody, can also detect very low concentrations of various analytes as the binding of the analyte to the gate region of the FET cause a change in the drain-source current.

Biosensors have a number of potential advantages over conventional binding assay. One important advantage is the ability to manufacture small-scale, but highly reproducible, biosensor units using microchip manufacturing methods.

There are many potential applications of biosensors of various types. The main requirements for a biosensor approach to be valuable in terms of research and commercial applications are the identification of a target molecule, availability of a suitable biological recognition element, and the potential for disposable portable detection biosensors to be preferred to sensitive laboratory-based techniques in some situations.

In one embodiment, the biosensor according to the present disclosure further comprises Zona pellucida 1 (ZP1), Zona pellucida 2 (ZP2), Zona pellucida 3 (ZP3) and/or an anti-IZUMO antibody or fragments thereof, wherein the ZP1, ZP2, ZP3, and/or the anti-IZUMO antibody or fragments thereof are immobilized on the substrate.

In one embodiment, the biosensor according to the present disclosure comprises the proteins JUNO and ZP3, wherein said proteins are immobilized on the substrate.

In one embodiment, the biosensor according to the present disclosure comprises the proteins JUNO and ZP2, wherein said proteins are immobilized on the substrate.

In one embodiment, the biosensor according to the present disclosure comprises the proteins JUNO and ZP1, wherein said proteins are immobilized on the substrate.

In one embodiment, the biosensor according to the present disclosure comprises the proteins JUNO and an anti-IZUMO antibody, wherein said proteins are immobilized on the substrate.

In one embodiment, the biosensor according to the present disclosure comprises the proteins JUNO, ZP2 and ZP3, wherein said proteins are immobilized on the substrate.

Presence of JUNO in combination with one or more of ZP1, ZP2, ZP3 and an anti-IZUMO antibody may improve the specificity of the biosensor and may allow a more precise determination of the sperm function of the analysed semen sample, and/or of the analysed sperm sample,. For example, presence of JUNO in combination with one or more of ZP1, ZP2, ZP3 and an anti-IZUMO antibody may allow a more precise determination of the capability of the sperm to bind the zona pellucida proteins, undergo acrosomal reaction and bind an oocyte.

In one embodiment, the proteins JUNO, ZP1, ZP2 and ZP3 which may be immobilized on the substrate are mammalian proteins. For example, the proteins JUNO, ZP1, ZP2 and ZP3 may be human proteins or fragments thereof. For example, the proteins JUNO, ZP1, ZP2 and ZP3 may be equine proteins or fragments thereof. For example, the proteins JUNO, ZP1, ZP2 and ZP3 may be canine proteins or fragments thereof. For example, the proteins JUNO, ZP1, ZP2 and ZP3 may be bovine proteins or fragments thereof.

In a further embodiment, the biosensor according to the present disclosure comprises the JUNO protein comprising or consisting of a polypeptide having at least 95% sequence identity, such as at least 96% sequence identity, such as at least 97% sequence identity, such as at least 98% sequence identity, such as at least 99% sequence identity entity, such as about 100% sequence identity to SEQ ID NO: 1 or an orthologue thereof, or a fragment of said protein.

In one embodiment the biosensor according to the present disclosure is provided, wherein the ZP1 comprises or consists of a polypeptide having at least 95% sequence identity, such as at least 96% sequence identity, such as at least 97% sequence identity, such as at least 98% sequence identity, such as at least 99% sequence identity entity, such as about 100% sequence identity to SEQ ID NO: 2 or an orthologue thereof, or a fragment of said protein.

In a further embodiment the biosensor according to the present disclosure is provided, wherein the ZP2 comprises or consists of a polypeptide having at least 95% sequence identity, such as at least 96% sequence identity, such as at least 97% sequence identity, such as at least 98% sequence identity, such as at least 99% sequence identity entity, such as about 100% sequence identity to SEQ ID NO: 3 or an orthologue thereof, or a fragment of said protein.

In a further embodiment the biosensor according to the present disclosure is provided, wherein the ZP3 comprises or consists of a polypeptide having at least 95% sequence identity, such as at least 96% sequence identity, such as at least 97% sequence identity, such as at least 98% sequence identity, such as at least 99% sequence identity entity, such as about 100% sequence identity to SEQ ID NO: 4 or an orthologue thereof, or a fragment of said protein.

In some embodiments, at least one of the JUNO protein, ZP1, ZP2, ZP3, and anti-IZUMO antibody is conjugated to an additional moiety. For example, said additional moiety may be a peptide or a label. For example, at least one of the JUNO protein, ZP1, ZP2, ZP3, and anti-IZUMO antibody is conjugated to a polyhistidine-tag.

In some embodiments, the biosensor according to the present disclosure is provided, wherein the substrate is microbeads, a dish, a chip or an electrode.

In one embodiment, the substrate is polymeric microbeads. For example, the substrate may be agarose, cellulose, nitrocellulose or latex microbeads. In some embodiments, the microbeads are configured such that it can be coupled to a microscope, an optical transducer or a measurement circuit.

In one embodiment, the substrate is a dish, such as a plastic dish, a ceramic dish or a glass dish. In a further embodiment, said dish is configured such that it can be coupled to a microscope or an optical transducer.

In some embodiments, the chip is a glass chip.

The term “glass” as used herein is equivalent to quartz or silica, comprising silicon and oxygen atoms in a continuous framework with an overall chemical formula of SiO₂.

In particular embodiments, the electrode is a carbon, gold or platinum electrode. In one embodiment, the electrode is a screen printed electrode.

In some embodiments, the substrate has a modified surface. In one embodiment, at least one surface of the substrate is coated with a layer of gold. In some embodiments, at least one surface of the substrate is modified with nanoparticles selected from the group consisting of gold, silver, copper oxide, graphene, iron oxide and combinations thereof.

In particular embodiments, the biosensor according to the present disclosure is configured such that the substrate can be coupled to a microscope, an electrochemical workstation, a surface plasmon resonance detector, a measurement circuit or an optical transducer.

In one embodiment, the substrate is an electrode and configured such that it can be coupled to an electrochemical workstation or a measurement circuit.

In one embodiment, the substrate is a chip and is configured such that it can be coupled to a surface plasmon resonance detector.

### Immobilization of Proteins

The present disclosure is directed to a biosensor

For detection and quantification of sperm function, said biosensor comprising a substrate and the protein JUNO, or a fragment thereof, immobilized on the substrate.

Immobilization of the biological element, such as the protein of interest on the surface of the sensor (be it metal, polymer or glass) is a necessary and critical step in the design of biosensors. Different immobilization techniques exist depending on the substrate employed, these techniques are known to the person skilled in the art.

In some embodiments the biosensor according to the present disclosure is provided, wherein at least one of the JUNO protein, ZP1, ZP2, ZP3, and anti-IZUMO antibody is conjugated to an additional moiety. In some embodiments, the additional moiety is a peptide. In one embodiment, the additional moiety is a label.

In some embodiments, the additional moiety is a peptide, for example a polyhistidine tag (His-tag).

In some embodiments, the additional moiety is a label, also referred to as a fluorescent tag or a probe.

The polyhistidine-tag can be successfully used for the immobilization of proteins on a surface such as on a metal surface, for example a gold-, nickel- or cobalt-coated microtiter plate or on a protein array.

In some embodiments the biosensor according to the present disclosure is provided, wherein the JUNO protein, ZP1, ZP2, ZP3 and/or the anti-IZUMO antibody is immobilized on the substrate.

In some embodiments the biosensor according to the present disclosure is provided, wherein the JUNO protein, ZP1, ZP2, ZP3 and/or the anti-IZUMO antibody is immobilized on the substrate via a nanoparticle, for example via a gold nanoparticle. The presence of a nanoparticle between the substrate and the protein is advantageous as it prevents unfolding of the protein and helps the protein to stay in the correct conformation.

### Methods

It is an aspect of the disclosure to provide a method for manufacturing a biosensor comprising a JUNO protein, such as a biosensor disclosed herein, the method comprising:
a. providing a substrate,
b. providing the JUNO protein,
c. immobilizing the JUNO protein on the substrate,
   thereby manufacturing a biosensor comprising the JUNO protein.

In some embodiments, the method for manufacturing a biosensor further comprises immobilizing on the substrate one or more of ZP1, ZP2, ZP3 and an anti-IZUMO antibody.

It is a further aspect of the disclosure to provide a method of selecting sperm, said method comprising:
a. Providing a semen sample from a subject,
b. Contacting the semen sample with the biosensor according to the present disclosure,
c. Visualizing a sperm bound to the biosensor by microscopy, and
d. Selecting sperm bound to the biosensor.

It is a further aspect of the disclosure to provide a method of selecting sperm, said method comprising:
a. Providing a sperm sample from a subject,
b. Contacting the sperm sample with the biosensor according to the present disclosure,
c. Visualizing a sperm bound to the biosensor by microscopy, and
d. Selecting sperm bound to the biosensor.

In one embodiment according to the method of the present disclosure, the semen sample comprises, or is suspected of comprising, one or more sperm cells.

### Home Device

It is also an aspect of the disclosure to provide a hand-held device for detection and/or quantification of sperm function, the device comprising:
a. An inlet for a sample;
b. A biosensor comprising a JUNO protein or a fragment thereof, wherein the JUNO protein is immobilized on the biosensor, and wherein the inlet is configured to place the sample in contact with the sensor;
c. A detector configured to receive a signal from the sensor and transform it into a format readable by a user;
d. Optionally, means for separating cellular components from the sample.

In particular embodiments, the hand-held device according to the present disclosure comprises the biosensor as defined in any one of the embodiments of the present disclosure.

### Subjects

It is an aspect of the disclosure to provide a method according to the present disclosure, wherein the subject is a human subject. In particular embodiments, the human subject is a child or an adult.

In further embodiments of the method according to the present disclosure, the subject is a mammal. In further embodiments of the method according to the present disclosure, the subject is a horse, cow, buffalo, sheep, pig, goat, cat or dog.

In some embodiments of the present disclosure, the subject is a horse, and the biosensor comprises a JUNO protein or a fragment thereof immobilized on the substrate, wherein said JUNO protein has a sequence having at least 95% sequence identity to an equine JUNO protein of SEQ ID NO: 6, such as at least 96% sequence identity, such as at least 97% sequence identity, such as at least 98% sequence identity, such as at least 98% sequence identity, such as at least 99% sequence identity, such as about 100% sequence identity to said an equine JUNO protein of SEQ ID NO: 6.

In some embodiments of the present disclosure, the subject is a dog, and the biosensor comprises a JUNO protein or a fragment thereof immobilized on the substrate, wherein said JUNO protein is a canine JUNO protein.

In some embodiments of the present disclosure, the subject is a cow, and the biosensor comprises a JUNO protein or a fragment thereof immobilized on the substrate, wherein said JUNO protein is a bovine JUNO protein.

### Sample

In particular embodiments of the methods according to the present disclosure, the sample is a semen sample, optionally wherein the sample has been treated prior to analysis.

In particular embodiments of the methods according to the present disclosure, the sample is a sperm sample, optionally wherein the sample has been treated prior to analysis.

In one embodiment according to the method of the present disclosure, the semen sample and/or the sperm sample comprises, or is suspected of comprising, one or more sperm cells.

In some embodiments, the semen sample is treated prior to analysis and said treatment comprises liquefaction of the sperm.

Semen sample may herein be used interchangeably with sperm sample and seminal fluid. A semen sample and/or a sperm sample is defined herein as a sample of seminal fluid that may contain spermatozoa. The semen sample may further, in specific embodiments of the present disclosure, be treated, such as diluted, by the use of solvents, mediums, buffers and/or fluids suitable for the treatment, such as dilution, of semen/sperm samples.

Semen is secreted by the gonads (sexual glands) and other sexual organs of male or hermaphroditic animals and can fertilize female ova. In humans, seminal fluid contains several components besides spermatozoa: proteolytic and other enzymes as well as fructose are elements of seminal fluid, which promote the survival of spermatozoa, and provide a medium through which they can move or “swim”. Semen is produced and originates from the seminal vesicle, which is located in the pelvis.

The term “sperm” has used herein refers to the male reproductive cell and is synonymous of “sperm cell”. A uniflagellar sperm cell that is motile is referred to as a spermatozoon, whereas a non-motile sperm cell is referred to as a spermatium. Within the present disclosure, the terms “sperm” and “spermatozoa” are used interchangeably.

### Detection Technologies

In some embodiments of the methods according to the present disclosure, the sperm function is detected using surface plasmon resonance (SPR). In particular embodiments, the surface plasmon resonance readout is used to determine the concentration of one or more of the thyroid hormones.

Surface plasmon resonance is the resonant oscillation of conduction electrons at the interface between negative and positive permittivity material stimulated by incident light. SPR is the basis of many standard tools for measuring adsorption of material onto planar metal (such as gold or silver) surfaces or onto the surface of metal nanoparticles. It is the fundamental principle behind many color-based biosensor applications, different sensors and diatom photosynthesis. SPR may be used to detect biomolecular binding interactions. In SPR, one molecular partner such as a protein is immobilized on a metallic film. Light excites surface plasmons in the metal; when the binding partner binds to the immobilized molecule, this causes a detectable change in the surface plasmon signal.

In some embodiments of the methods according to the present disclosure, the sperm function is detected or quantified by electrochemical transduction.

Electrochemical biosensors, also referred to as biosensors utilizing electrochemical transduction provide an attractive means to analyze the content of a biological sample due to the direct conversion of a biological event to an electronic signal. The most common techniques in electrochemical biosensing comprise cyclic voltammetry, chronoamperometry, chronopotentiometry, impedance spectroscopy, and field-effect transistor based methods along with nanowire or magnetic nanoparticle-based biosensing. Additional measurement techniques useful in combination with electrochemical detection may further comprise the electrochemical versions of surface plasmon resonance, optical waveguide lightmode spectroscopy, ellipsometry, quartz crystal microbalance, and scanning probe microscopy.

### Male Infertility

Male infertility refers to a male's inability to cause pregnancy in a fertile female. In humans it accounts for 40-50% of infertility. It affects approximately 7% of all men. Male infertility is commonly due to deficiencies in the semen, and semen quality is used as a surrogate measure of male fecundity.

Male infertility is defined as the inability of a male individual to cause pregnancy in a fertile female individual. A cause of male infertility may be the production of non-functional sperm.

A diagnosis of male infertility may be based on a battery of functional tests, for example on any one of the sperm functions tests disclosed herein. The tests may be evaluated according to guidelines known to the person of skill in the art, for example as described in the WHO laboratory manual for the Examination and processing of human semen, WHO, 5^{th} Edition, 2010, ISBN 978 92 4 154778 9 (see in particular Chapter 4).

Sequences

SEQ ID NO: 1: JUNO_HUMAN Sperm-egg fusion protein
Juno
MACWWPLLLELWTVMPTWAGDELLNICMNAKHHKRVPSPEDKLYEECIPW
KDNACCTLTTSWEAHLDVSPLYNFSLFHCGLLMPGCRKHFIQAICFYECS
PNLGPWIQPVGSLGWEVAPSGQGERVVNVPLCQEDCEEWWEDCRMSYTCK
SNWRGGWDWSQGKNRCPKGAQCLPFSHYFPTPADLCEKTWSNSFKASPER
RNSGRCLQKWFEPAQGNPNVAVARLFASSAPSWELSYTIMVCSLFLPFLS
SEQ ID NO: 2: ZP1_HUMAN Zona pellucida sperm-
binding protein 1
MAGGSATTWGYPVALLLLVATLGLGRWLQPDPGLPGLRHSYDCGIKGMQL
LVFPRPGQTLRFKVVDEFGNRFDVNNCSICYHWVTSRPQEPAVFSADYRG
CHVLEKDGRFHLRVFMEAVLPNGRVDVAQDATLICPKPDPSRTLDSQLAP
PAMFSVSTPQTLSFLPTSGHTSQGSGHAFPSPLDPGHSSVHPTPALPSPG
PGPTLATLAQPHWGTLEHWDVNKRDYIGTHLSQEQCQVASGHLPCIVRRT
SKEACQQAGCCYDNTREVPCYYGNTATVQCFRDGYFVLVVSQEMALTHRI
TLANIHLAYAPTSCSPTQHTEAFVVFYFPLTHCGTTMQVAGDQLIYENWL
VSGIHIQKGPQGSITRDSTFQLHVRCVFNASDFLPIQASIFPPPSPAPMT
QPGPLRLELRIAKDETFSSYYGEDDYPIVRLLREPVHVEVRLLQRTDPNL
VLLLHQCWGAPSANPFQQPQWPILSDGCPFKGDSYRTQMVALDGATPFQS
HYQRFTVATFALLDSGSQRALRGLVYLFCSTSACHTSGLETCSTACSTGT
TRQRRSSGHRNDTARPQDIVSSPGPVGFEDSYGQEPTLGPTDSNGNSSLR
PLLWAVLLLPAVALVLGFGVFVGLSQTWAQKLWESNRQ
SEQ ID NO: 3: ZP2_HUMAN Zona pellucida sperm-
binding protein 2
MACRQRGGSWSPSGWFNAGWSTYRSISLFFALVTSGNSIDVSQLVNPAFP
GTVTCDEREITVEFPSSPGTKKWHASVVDPLGLDMPNCTYILDPEKLTLR
ATYDNCTRRVHGGHQMTIRVMNNSAALRHGAVMYQFFCPAMQVEETQGLS
ASTICQKDFMSFSLPRVFSGLADDSKGTKVQMGWSIEVGDGARAKTLTLP
EAMKEGFSLLIDNHRMTFHVPFNATGVTHYVQGNSHLYMVSLKLTFISPG
QKVIFSSQAICAPDPVTCNATHMTLTIPEFPGKLKSVSFENQNIDVSQLH
DNGIDLEATNGMKLHFSKTLLKTKLSEKCLLHQFYLASLKLTFLLRPETV
SMVIYPECLCESPVSIVTGELCTQDGFMDVEVYSYQTQPALDLGTLRVGN
SSCQPVFEAQSQGLVRFHIPLNGCGTRYKFEDDKVVYENEIHALWTDFPP
SKISRDSEFRMTVKCSYSRNDMLLNINVESLTPPVASVKLGPFTLILQSY
PDNSYQQPYGENEYPLVRFLRQPIYMEVRVLNRDDPNIKLVLDDCWATST
MDPDSFPQWNVVVDGCAYDLDNYQTTFHPVGSSVTHPDHYQRFDMKAFAF
VSEAHVLSSLVYFHCSALICNRLSPDSPLCSVTCPVSSRHRRATGATEAE
KMTVSLPGPILLLSDDSSFRGVGSSDLKASGSSGEKSRSETGEEVGSRGA
MDTKGHKTAGDVGSKAVAAVAAFAGVVATLGFIYYLYEKRTVSNH
SEQ ID NO: 4: ZP3_HUMAN Zona pellucida sperm-
binding protein 3
MELSYRLFICLLLWGSTELCYPQPLWLLQGGASHPETSVQPVLVECQEAT
LMVMVSKDLFGTGKLIRAADLTLGPEACEPLVSMDTEDVVRFEVGLHECG
NSMQVTDDALVYSTFLLHDPRPVGNLSIVRTNRAEIPIECRYPRQGNVSS
QAILPTWLPFRTTVFSEEKLTFSLRLMEENWNAEKRSPTFHLGDAAHLQA
EIHTGSHVPLRLFVDHCVATPTPDQNASPYHTIVDFHGCLVDGLTDASSA
FKVPRPGPDTLQFTVDVFHFANDSRNMIYITCHLKVTLAEQDPDELNKAC
SFSKPSNSWFPVEGSADICQCCNKGDCGTPSHSRRQPHVMSQWSRSASRN
RRHVTEEADVTVGPLIFLDRRGDHEVEQWALPSDTSVVLLGVGLAVVVSL
TLTAVILVLTRRCRTASHPVSASE
SEQ ID NO: 5: IZUMO1_HUMAN Izumo sperm-egg fusion
protein 1
MGPHFTLLCAALAGCLLPAEGCVICDPSVVLALKSLEKDYLPGHLDAKHH
KAMMERVENAVKDFQELSLNEDAYMGVVDEATLQKGSWSLLKDLKRITDS
DVKGDLFVKELFWMLHLQKETFATYVARFQKEAYCPNKCGVMLQTLIWCK
NCKKEVHACRKSYDCGERNVEVPQMEDMILDCELNWHQASEGLTDYSFYR
VWGNNTETLVSKGKEATLTKPMVGPEDAGSYRCELGSVNSSPATIINFHV
TVLPKMIKEEKPSPNIVTPGEATTESSISLQPLQPEKMLASRLLGLLICG
SLALITGLTFAIFRRRKVIDFIKSSLFGLGSGAAEQTQVPKEKATDSRQQ

### EXAMPLES

### Example 1. Electrochemical Biosensor for Sperm Binding Employing Cyclic Voltammetry

The biosensors were fabricated on a screen-printed gold electrode. Briefly, 10 μL of 2 mg/mL cysteamine hydrochloride was added on the gold electrode and allowed to dry in dark at room temperature. The electrode was washed with deionized water followed by modification with 10 μL of citrate-capped gold nanoparticles. After drying and washing with deionized water, 10 μL of 50 mM nitrilotriacetic acid was added and incubated overnight at room temperature. This was followed by blocking with 1% bovine serum albumin in 0.01 M phosphate buffer saline at pH 7.4. 10 μL of 10 mM nickel sulfate was added and incubated at room temperature for 2 hours. After washing the electrode with 0.01 M phosphate buffer saline at pH 7.4, 0.5 μg of JUNO or ZP3 containing a polyhistidine tag were immobilized on the biosensor.

Semen samples were allowed to liquefy following standard procedures and diluted to different dilutions in a semen preparation medium (#1070/1069, Origio A/S Denmark, https://origio.marketport.net/MarketingZone/MZDirect/Source/510e96f4-075b-4684-9a99-d472c1e3d31b). The biosensor was connected to an electrochemical station, diluted semen sample was added on the biosensor, and the cyclic voltammetric response of the interaction between the immobilized proteins and the sperms present in the semen sample was recorded.

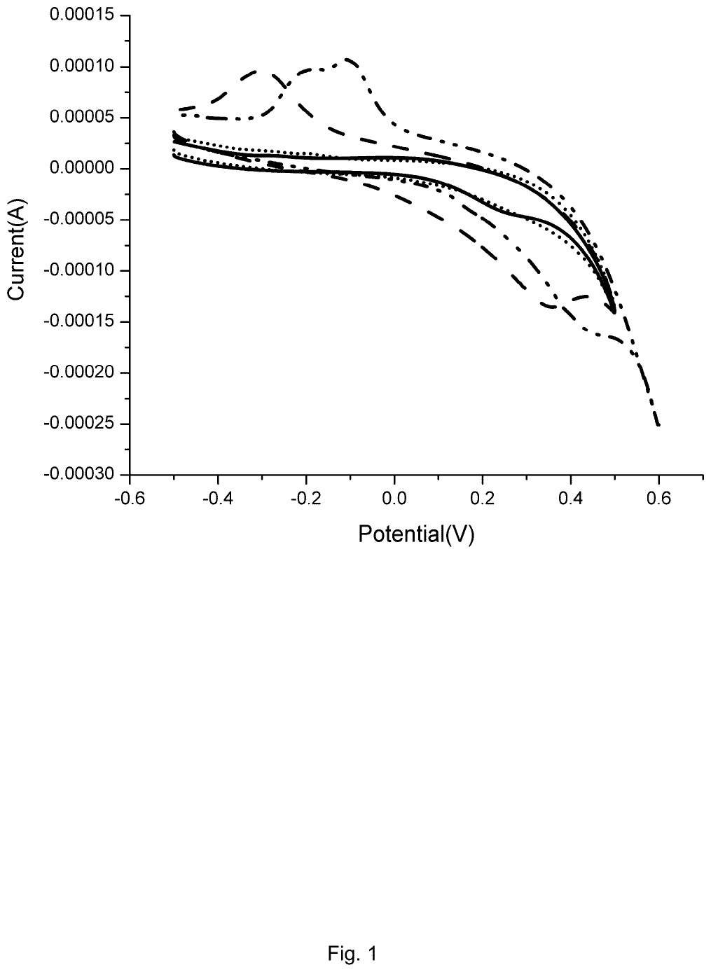

The results are shown in FIG. 1-FIG. 3, wherein FIG. 1 shows the cyclic voltammetric response of a) Electrode-gold nanoparticles-BSAblocking after addition of buffer only (solid line) and after addition of semen sample diluted 1.67×10⁻²X in buffer (dotted line) b) Electrode-gold nanoparticles-JUNO-BSAblocking (dash dot dot line) and c) Electrode-gold nanoparticles-ZP3-BSAblocking response (dashed line) after addition of semen sample diluted 1.67×10⁻²X in buffer, with the dilution factor given by the initial volume/final volume.

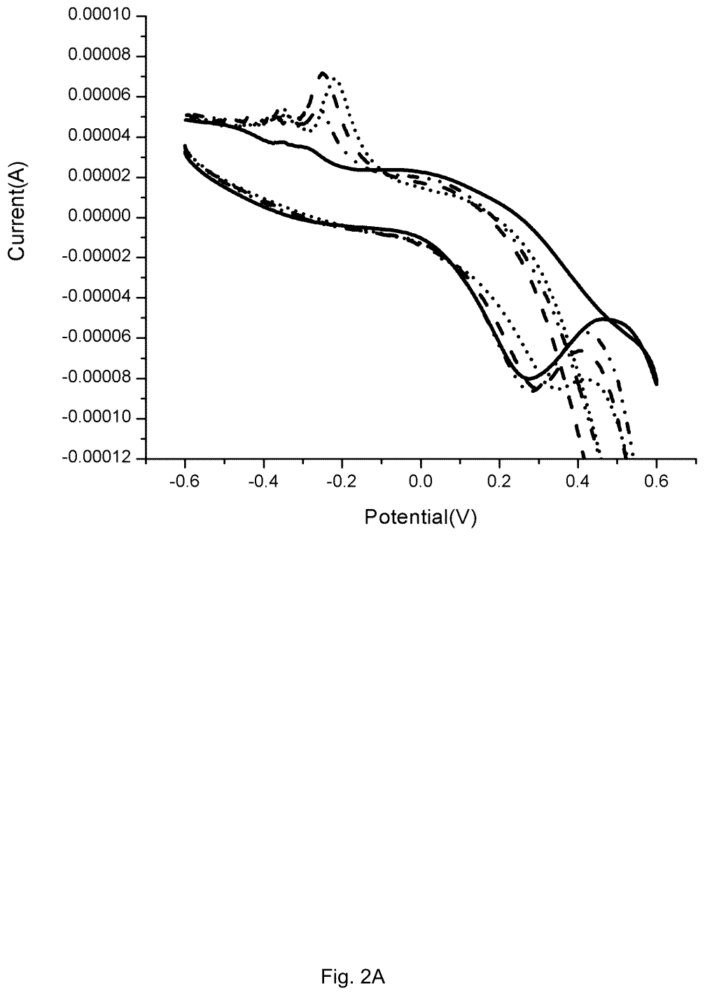

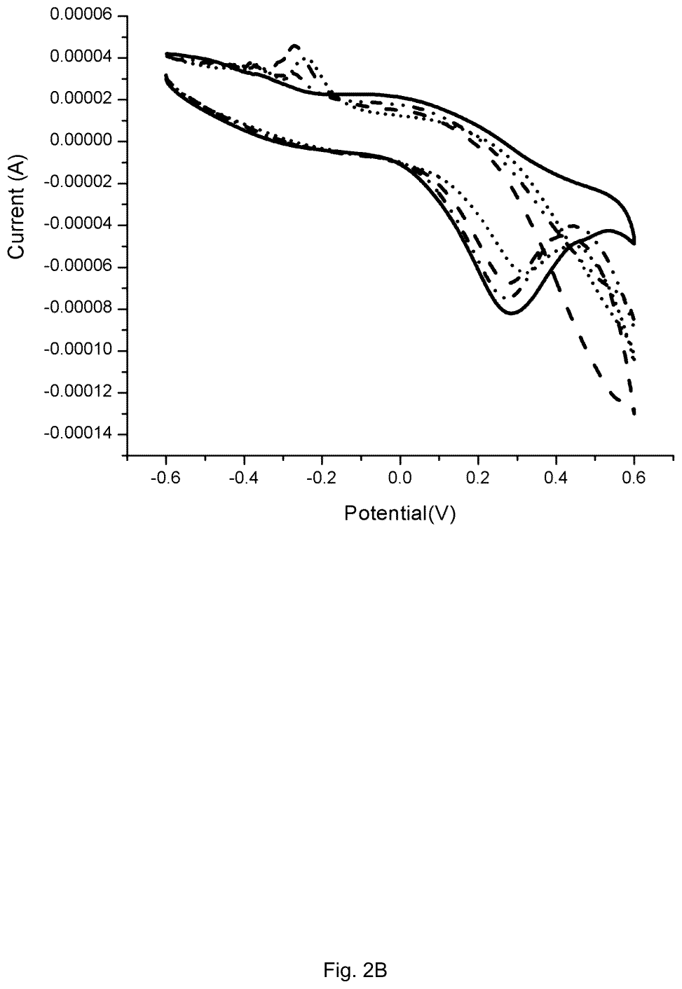

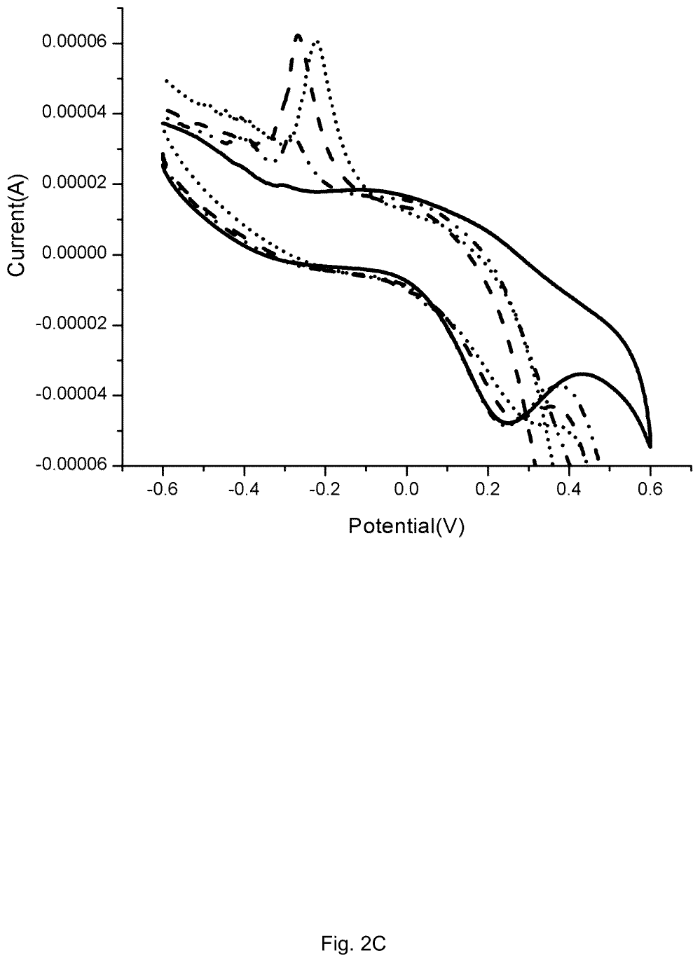

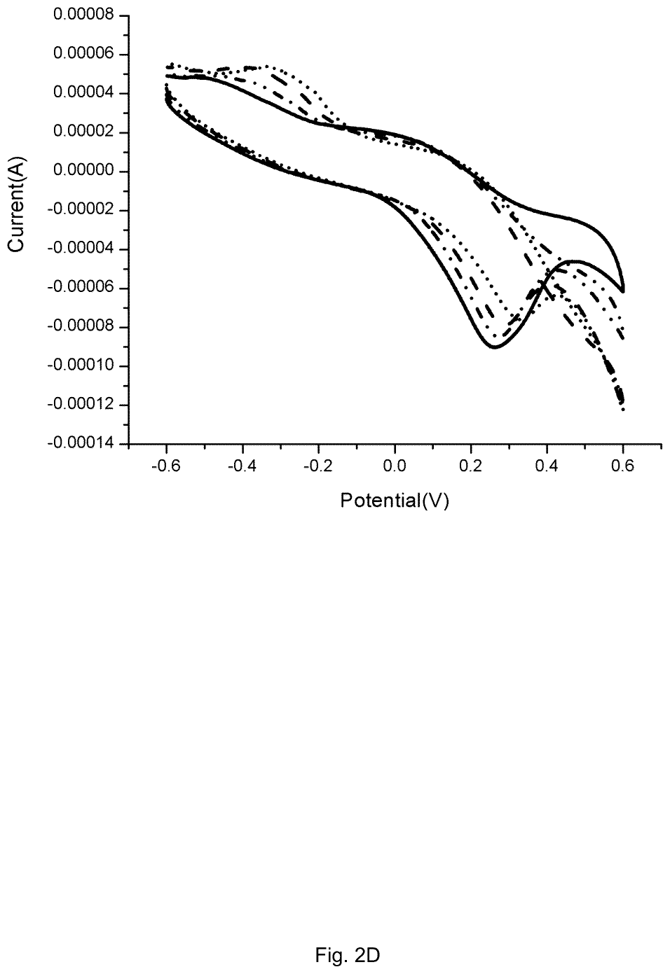

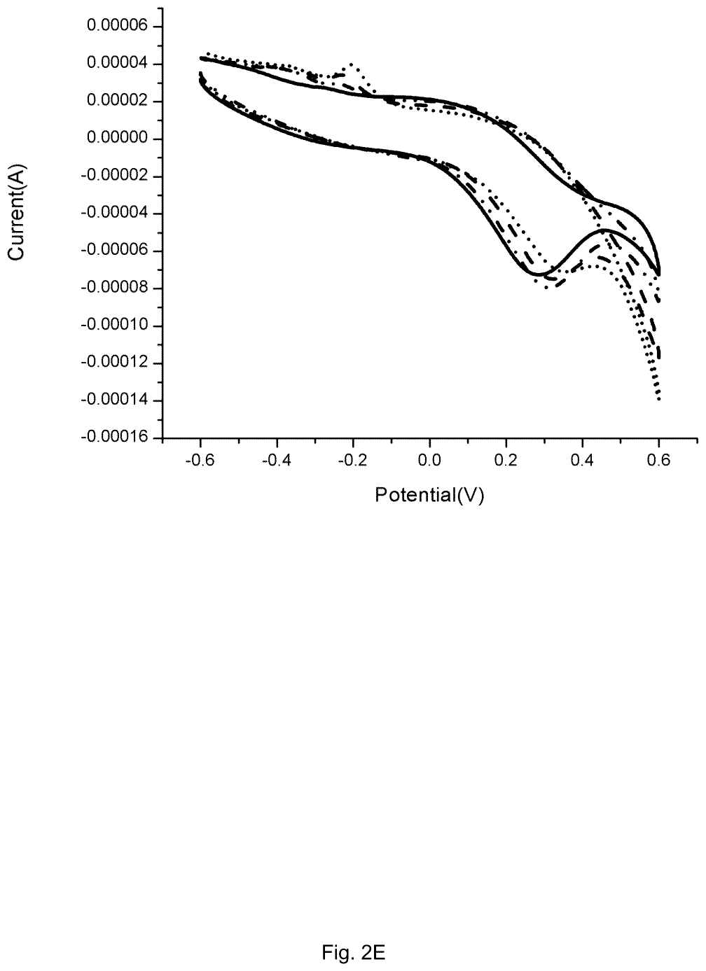

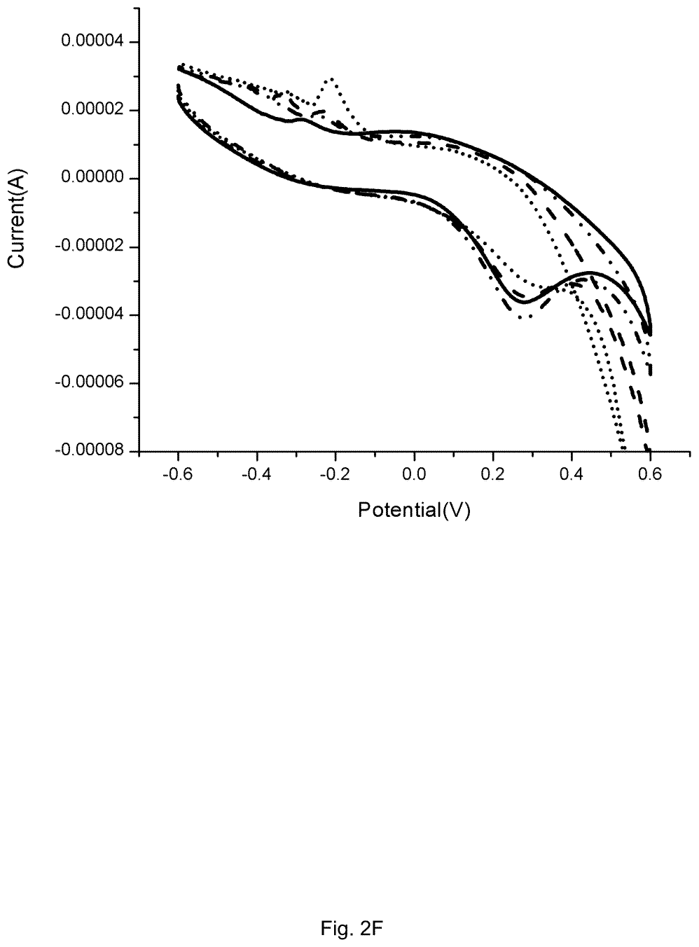

FIG. 2 shows cyclic voltammetric response of Electrode-gold nanoparticles-ZP3-BSAblocking after addition of three different dilutions (1.56×10⁻⁵X (dotted line), 1.67×10⁻²X (dash dot dot line) and 2.5×10⁻²X (dashed line)) of 6 different semen samples (FIG. 2A-F), while solid line shows electrode response with buffer only (no semen sample).

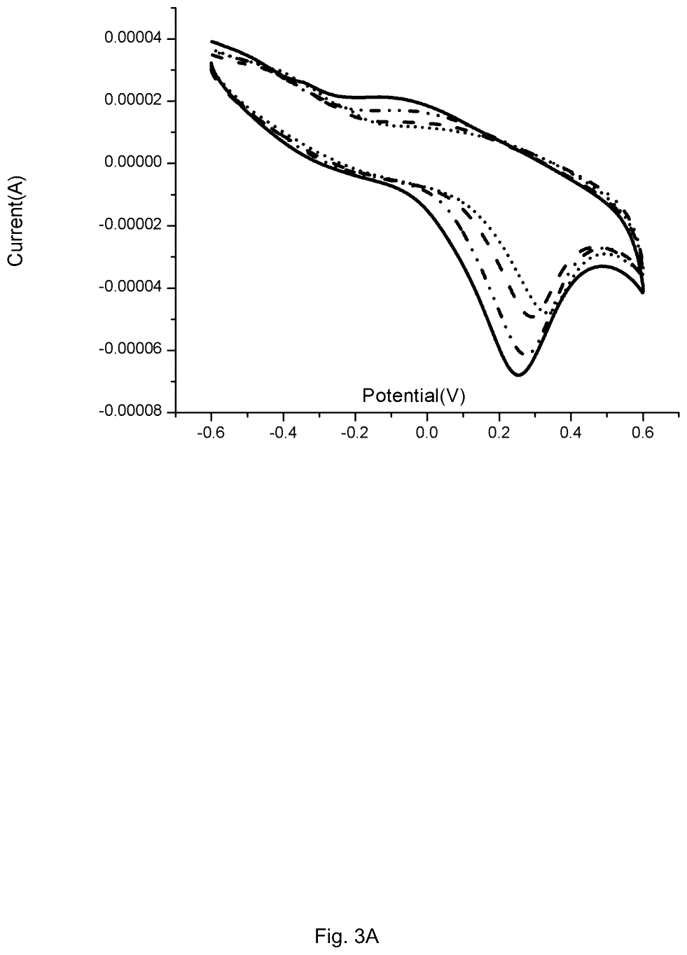

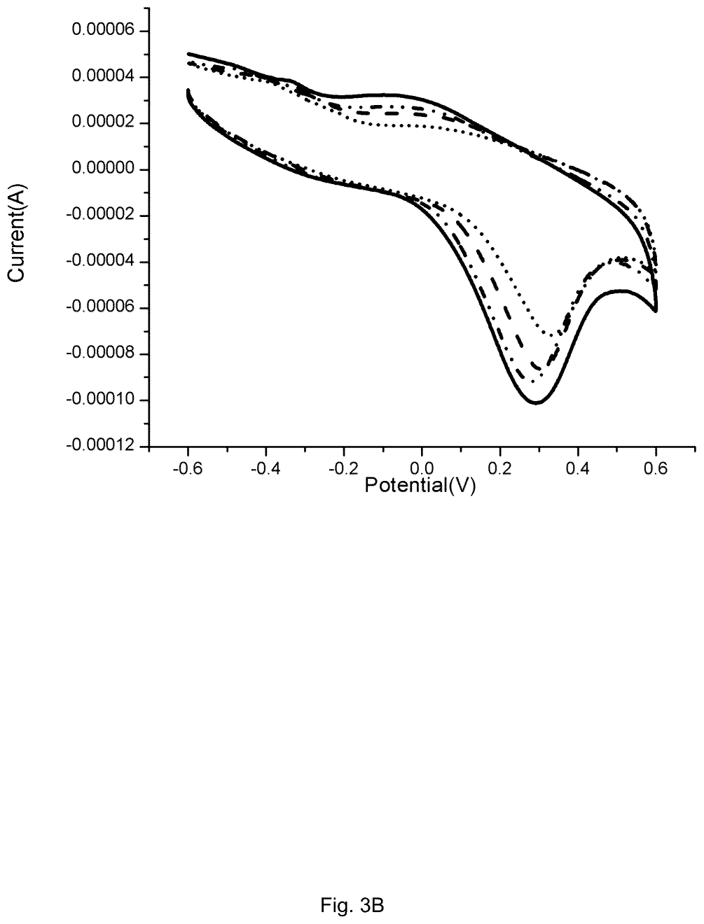

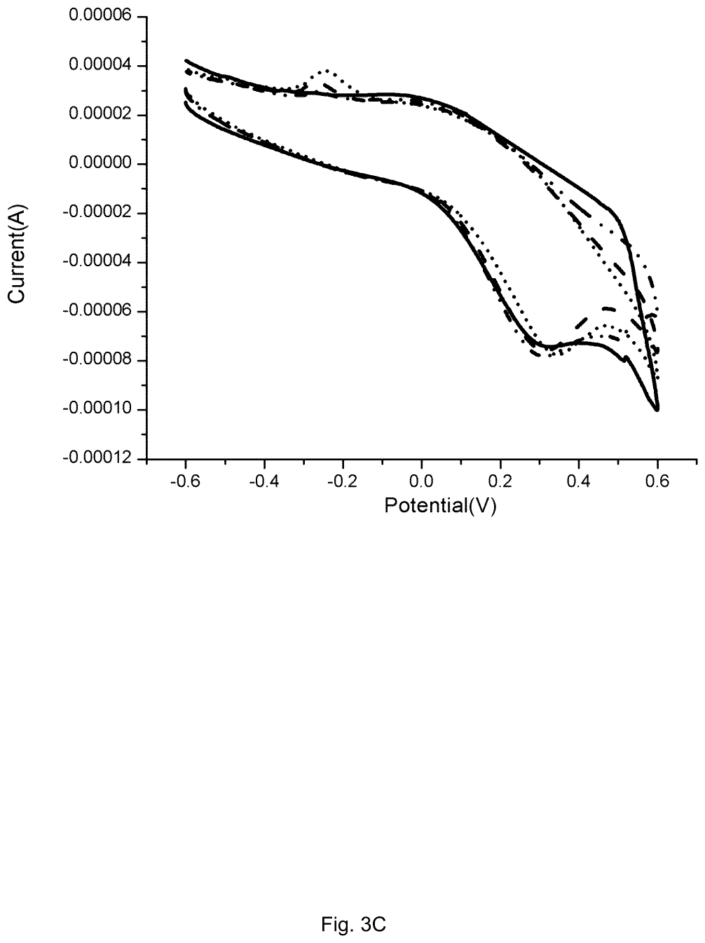

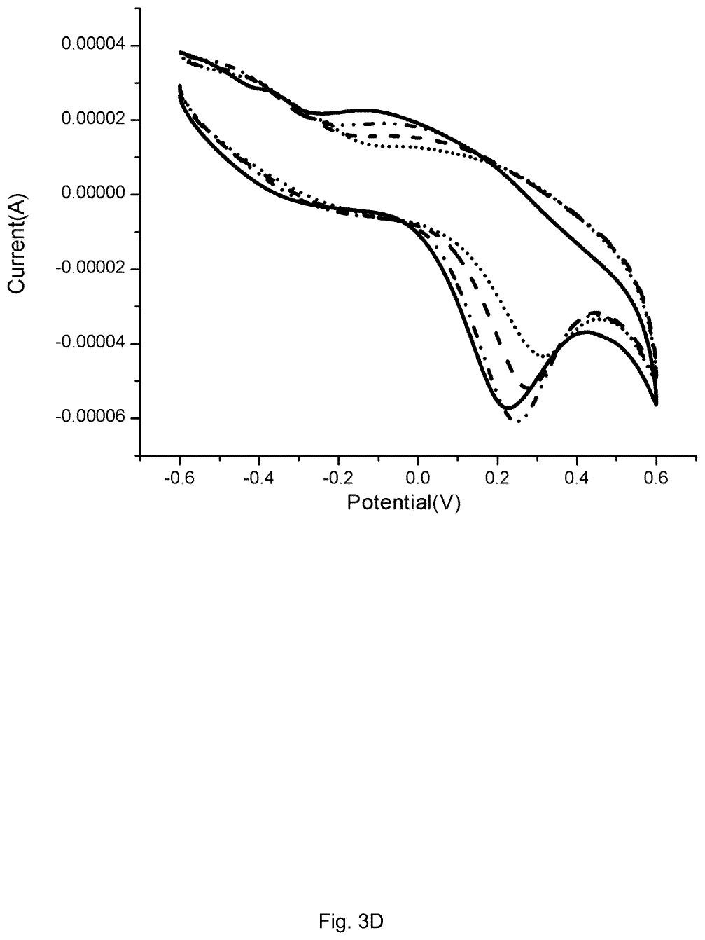

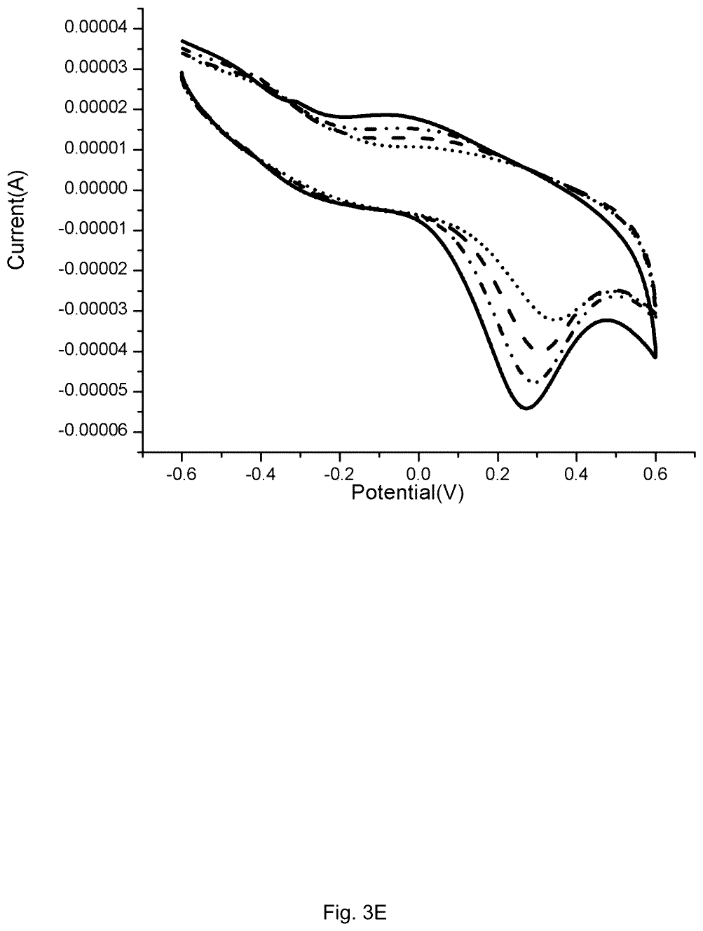

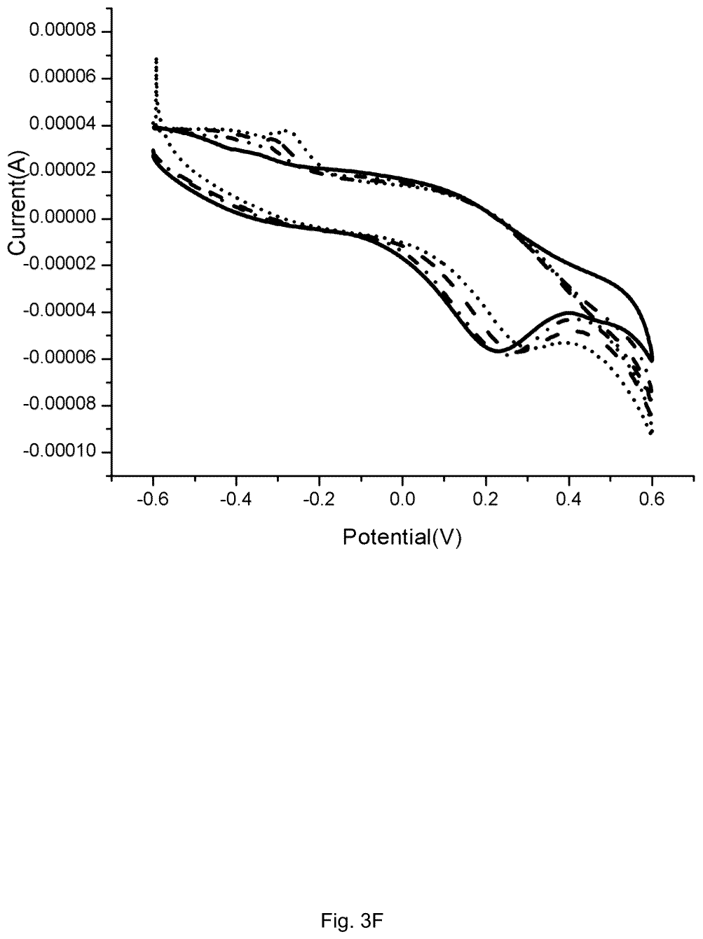

FIG. 3 shows cyclic voltammetric response of Electrode-gold nanoparticles-JUNO-BSAblocking after addition of three different dilutions (1.56×10⁻⁵X (dotted line), 1.67×10⁻²X (dash dot dot line) and 2.5×10⁻²X (dashed line)) of semen samples S1, S2, S3, S4, S5 and S6 (FIG. 3A-F, respectively), while solid line shows electrode response with buffer only (no semen sample).

### Conclusion

As shown in FIG. 1, sperms do not bind to a biosensor without JUNO or ZP3 immobilized. In presence of JUNO or ZP3, the spermatozoa interact with the biosensor as evident from the distinct reduction and/or oxidation peaks in the cyclic voltammetric curves. Further, the binding is quantifiable using cyclic voltammetry. For both, ZP3-spermatozoa and JUNO-spermatozoa interactions, the reduction and oxidation peaks shift in a trend consistent with the number of spermatozoa available for the binding with ZP3 and JUNO, as shown in FIG. 2 and FIG. 3, respectively. Further, as the signal remains unchanged between null and non-zero sperm concentrations for negative control, i.e. upon absence of the binding proteins (FIG. 1), the probed interactions are protein-specific.

### Example 2. Electrochemical Biosensor for Sperm Binding Analysed Employing Electro-Impedance Spectroscopy

Screen printed gold electrodes are functionalized via 10 μl of 2 mg/ml Cysteamine hydrochloride (CyHCI) and incubated in dark for 2-4 h at room temperature. After washing the electrode with deionized water, 5 μl of Asn-AuNPs are dispensed onto the functionalized electrode. The electrode is incubated for 5 h at room temperature and then washed with deionized water. Further 5 μl of 160 μg/ml of JUNO and 160 μg/ml ZP3 in 5μl PB (10 mM, pH 7.4) is added and incubated overnight at 4° C. Finally the electrode is blocked with 10 μl of 1% BSA in PBS for 5 h at 4° C.

The modified electrode is connected to an electrochemical workstation.

5-50 μL of pre-treated semen sample is added on to the working electrode, or the electrode is immersed in the sample vial. The sperm cells are allowed to bind to the modified electrode for 1-10 minutes, followed by washing with deionized water. The binding is analysed via electro-impedance spectroscopy in presence of suitable concentrations of a redox mediator.

Similar experiments are performed using JUNO only, ZP2 and combination of ZP2, ZP3, JUNO.

For this and all the following examples, the semen sample is pre-treated (liquefied) prior to analysis according to the following procedure:
The semen sample collected in the laboratory or clinic is allowed to liquefy at room temperature or in an incubator at 37° C. for 15-60 minutes. This may be assisted by gentle rotation or stirring to prevent heterogeneously liquefied semen sample.
This liquefied semen sample may undergo washing, reconstitution or dilution with a suitable semen preparation medium.

### Conclusion

A sensor comprising screen printed gold electrodes functionalized and modified with JUNO, or a combination of JUNO and any one of ZP2 and ZP3 or combinations thereof, can be used to detect and quantify sperm function via an electrochemical workstation.

### Example 3. Electrochemical Biosensor for Acrosomal Reaction

Screen printed gold electrodes are functionalized via 10 μl of 2 mg/ml Cysteamine hydrochloride (CyHCl) and incubated in dark for 2-4 h at room temperature. After washing the electrode with deionized water, 5 μl of Asn-AuNPs are dispensed onto the functionalized electrode. The electrode is then incubated for 5 h at room temperature and then washed with deionized water. Further 5 μl of 160 μg/ml of JUNO and 160 μg/ml ZP3 in 5 μl PB (10 mM, pH 7.4) is added and incubated overnight at 4° C. Finally the electrode is blocked with 10 μl of 1% BSA in PBS for 5 h at 4° C.

The modified electrode is connected to an electrochemical workstation.

5-50 μL of pre-treated semen sample is added on to the working electrode, or the electrode is immersed in the sample vial. The sperm cells are allowed to bind to the modified electrode while the binding is analysed electrochemically via electro-impedance spectroscopy or amperometry.

Similar experiments are performed using ZP2 and combination of JUNO, ZP3, ZP2.

### Conclusion

A sensor comprising screen printed gold electrodes functionalized and modified with JUNO, or a combination of JUNO and any one of ZP2 and ZP3 or combinations thereof, can be used to detect and quantify sperm function via an electrochemical workstation.

### Example 4. Detection/Quantification of Sperm Function Via Surface Plasmon Resonance (SPR)

SPR chips are chemically functionalized and modified with any one of ZP2, ZP3 and JUNO, or combinations thereof. The chips are then washed with deionized water, and the uncoated area is blocked with bovine serum albumin (BSA). The chips are used in conjunction with an SPR detector. A flow of a pre-treated semen sample is let over the chip's surface. The interaction between the protein coat and the sperm cells is analyzed as a mass change in terms of binding kinetics and affinity. Sperm function is thereby detected and quantified.

### Conclusion

A sensor comprising SPR chips are chemically functionalized and modified with any one of ZP2, ZP3 and JUNO, or combinations thereof can be used to detect and quantify sperm function via an SPR detector.

### Example 5. Detection/Quantification of Sperm Binding Via an Optical Transducer

Whole or a part of a glass dish is chemically functionalized and modified first with AuNPs and then with any one of ZP2, ZP3 and JUNO, or combinations thereof. The dish is washed with deionized water, and the uncoated area is blocked with BSA. A drop of pre-treated semen sample is added on to the coated area, and visualized for binding under an optical transducer such as a bright field or dark field microscope. Sperm function is thereby detected and quantified.

### Conclusion

A sensor comprising any one of ZP2 and ZP3, or combinations thereof conjugated with AuNPs or AuNPs modified glass or plastic dish can be used to detect and quantify sperm function via an optical transducer.

### Example 6. Detection/Quantification of Acrosomal Reaction Via an Optical Transducer

The pre-treated semen sample is incubated with any one of ZP2 and ZP3, or combinations thereof, which have previously been conjugated with AuNPs. The sperms are allowed to interact with the proteins for 5-60 minutes, followed by washing via centrifugation. The sperms are then stained for acrosomal reaction with Pisum sativum agglutinin (PSA) labelled with fluorescein isothiocyanate (FITC) (PSA-FITC), and then visualized under an optical transducer such as a fluorescence microscope or a flow cytometer (see section 4.4.1 in http://apps.who.int/iris/bitstream/handle/10665/44261/9789241547789_eng.pdf;jsessionid=2EF9F9030760BB60B84C83708999AF64?sequence=1). Sperm function is thereby detected and quantified.

Alternatively, the pre-treated semen sample is incubated with any one of ZP2 and ZP3, or combinations thereof which have previously been conjugated with AuNP modified glass or plastic dish, for 5-60 minutes. The sperms are removed from the surface and then stained for acrosomal reaction with PSA-FITC, and then visualized under an optical transducer such as a fluorescence microscope or a flow cytometer. Sperm function is thereby detected and quantified.

Alternatively, the pre-treated semen sample is incubated with any one of ZP2 and ZP3, or combinations thereof, which have previously been conjugated with AuNP modified glass or plastic dish, for 5-60 minutes. The sperms are then labelled with anti-CD46 antibodies in situ or after removal from the substrate to detect sperms that have undergone acrosomal reaction. The detection is performed via an optical transducer such as a fluorescence microscope or a flow cytometer. Sperm function is thereby detected and quantified.

### Conclusion

A sensor comprising any one of ZP2, ZP3 and JUNO, or combinations thereof conjugated with AuNPs or AuNPs modified glass or plastic dish can be used to detected and quantify sperm function via an optical transducer.

1.-54. (canceled)

55. A biosensor for detection or quantification of sperm binding function, the biosensor comprising a substrate and an Izumo sperm-egg fusion 1 receptor (JUNO) protein or a fragment thereof (first fragment), wherein the JUNO protein or the first fragment is immobilized on the substrate, and wherein the JUNO protein or the first fragment, when immobilized on the substrate, binds a sperm, wherein at least a portion of the sperm comprises an IZUMO1 surface antigen.

56. The biosensor according to claim 55, further comprising Zona pellucida 1 (ZP1), Zona pellucida 2 (ZP2), Zona pellucida 3 (ZP3), an anti-IZUMO antibody, or a fragment of the ZP1, ZP2, ZP3, or the anti-IZUMO antibody (second fragment) immobilized on the substrate, wherein said second fragment is capable of binding to a sperm or to IZUMO.

57. The biosensor according to claim 55, wherein the proteins JUNO, ZP1, ZP2 and ZP3 are mammalian proteins.

58. The biosensor according to claim 55, wherein the JUNO protein comprises or consists of a polypeptide having at least 95% sequence identity to a protein of SEQ ID NO: 1 or an orthologue thereof, or a fragment of said protein.

59. The biosensor according to claim 55, wherein the JUNO protein or the first fragment thereof is conjugated to an additional moiety selected from a peptide and a label.

60. The biosensor according to claim 55, wherein the sperm binding function is determined from the binding of at least a portion of the sperm cell to the JUNO protein or first fragment immobilized on the substrate.

61. The biosensor according to claim 55,
wherein the substrate is a microbead and said microbead is coupled to a microscope, an optical transducer or a measurement circuit; or
wherein the substrate is a dish and said dish is coupled to a microscope or an optical transducer; or
wherein the substrate is a chip and said chip is coupled to a surface plasmon resonance detector; or
wherein the substrate is an electrode and said electrode is coupled to an electrochemical workstation or a measurement circuit.

62. The biosensor according to claim 55, wherein at least a portion of the substrate is modified with nanoparticles selected from gold, silver, copper oxide, graphene, iron oxide and combinations thereof.

63. The biosensor according to claim 55, wherein the biosensor is configured for detection or quantification of sperm binding function, wherein said sperm is in a semen sample.

64. A method for detecting or quantifying sperm binding function, wherein the method comprises the steps of:
a) Providing a semen sample from a subject, wherein said semen sample comprises one or more sperm cells,
b) Binding any sperm cells in the sample that exhibit IZUMO1 as a surface antigen by contacting the semen sample with the biosensor according to claim 55, and
c) Detecting or quantifying any sperm cells bound to the JUNO protein or the fragment thereof that is immobilized on the substrate,
thereby detecting or quantifying the sperm binding function of said sample.

65. A method of using a biosensor according to claim 55 to diagnose male infertility in a subject, wherein the method comprises the steps of:
a) Contacting a semen sample obtained from the subject with said biosensor, and
b) Detecting or quantifying any sperm in said sample that bind the JUNO protein or the fragment thereof that is immobilized on the substrate to diagnose if the subject is infertile.

66. The method according to claim 65, further comprising a step of treating said male infertility, wherein the treatment comprises administration of a medicament in a therapeutically effective amount or an artificial reproductive technology (ART).

67. The method according to claim 64, wherein the sperm binding function of said sample is quantified by determining the percentage of bound versus unbound sperm cells by microscopic analysis, electrochemical detection or surface plasmon resonance.

68. The method according to claim 64, wherein the sperm binding function of said sample is quantified by determining the acrosomal status of the sperm cells in the semen sample by microscopic analysis, electrochemical detection or surface plasmon resonance.

69. A method of selecting sperm, said method comprising:
a) Providing a semen sample from a subject, wherein said semen sample comprises one or more sperm cells,
b) Contacting the semen sample with the biosensor according to claim 55,
c) Visualizing a sperm cell bound to the biosensor by microscopy, and
d) Selecting at least one sperm cell bound to the biosensor.

70. A method for manufacturing a biosensor comprising a JUNO protein according to claim 55, the method comprising:
a) Providing a substrate,
b) providing the JUNO protein,
c) immobilizing the JUNO protein on the substrate, and
d) providing means for determining binding of a sperm cell to the JUNO protein immobilized on the substrate,
thereby manufacturing a biosensor comprising the JUNO protein.

71. The method according to claim 70,
wherein the substrate is a microbead and said microbead is coupled to a microscope, an optical transducer or a measurement circuit; or
wherein the substrate is a dish and said dish is coupled to a microscope or an optical transducer; or
wherein the substrate is a chip and said chip is coupled to a surface plasmon resonance detector; or
wherein the substrate is an electrode and said electrode is coupled to an electrochemical workstation or a measurement circuit.

72. The method according to claim 70, wherein the method further comprises immobilizing on the substrate one or more of ZP1, ZP2, ZP3 and an anti-IZUMO antibody.

73. A hand-held device for detection or quantification of sperm binding function, the device comprising:
a) An inlet for a sample;
b) A biosensor for detection or quantification of sperm binding function, the biosensor comprising a substrate and an Izumo sperm-egg fusion 1 receptor (JUNO) protein or a fragment thereof (first fragment), wherein the JUNO protein or the first fragment is immobilized on the substrate, wherein the JUNO protein or the first fragment, when immobilized on the substrate, binds a sperm, wherein at least a portion of the sperm comprises an IZUMO1 surface antigen, and wherein the inlet is configured to place the sample in contact with the substrate;
c) A detector configured to receive a signal from the substrate and transform it into a format readable by a user; and
d) Optionally, means for separating cellular components from the sample.

74. The hand-held device according to claim 73,
wherein the substrate is a microbead and said microbead is coupled to a microscope, an optical transducer or a measurement circuit; or
wherein the substrate is a dish and said dish is coupled to a microscope or an optical transducer; or
wherein the substrate is a chip and said chip is coupled to a surface plasmon resonance detector; or
wherein the substrate is an electrode and said electrode is coupled to an electrochemical workstation or a measurement circuit.


SEQUENCE LISTING (5 sequences)

SEQ ID NO: 1 - protein, 250 aa, Homo sapiens
MACWWPLLLELWTVMPTWAGDELLNICMNAKHHKRVPSPEDKLYEECIPWKDNACCTLTTSWEAHLDVSPLYNFSLFHCGLLMPGCRKHFIQAICFYECSPNLGPWIQPVGSLGWEVAPSGQGERVVNVPLCQEDCEEWWEDCRMSYTCKSNWRGGWDWSQGKNRCPKGAQCLPFSHYFPTPADLCEKTWSNSFKASPERRNSGRCLQKWFEPAQGNPNVAVARLFASSAPSWELSYTIMVCSLFLPFLS

SEQ ID NO: 2 - protein, 638 aa, Homo sapiens
MAGGSATTWGYPVALLLLVATLGLGRWLQPDPGLPGLRHSYDCGIKGMQLLVFPRPGQTLRFKVVDEFGNRFDVNNCSICYHWVTSRPQEPAVFSADYRGCHVLEKDGRFHLRVFMEAVLPNGRVDVAQDATLICPKPDPSRTLDSQLAPPAMFSVSTPQTLSFLPTSGHTSQGSGHAFPSPLDPGHSSVHPTPALPSPGPGPTLATLAQPHWGTLEHWDVNKRDYIGTHLSQEQCQVASGHLPCIVRRTSKEACQQAGCCYDNTREVPCYYGNTATVQCFRDGYFVLVVSQEMALTHRITLANIHLAYAPTSCSPTQHTEAFVVFYFPLTHCGTTMQVAGDQLIYENWLVSGIHIQKGPQGSITRDSTFQLHVRCVFNASDFLPIQASIFPPPSPAPMTQPGPLRLELRIAKDETFSSYYGEDDYPIVRLLREPVHVEVRLLQRTDPNLVLLLHQCWGAPSANPFQQPQWPILSDGCPFKGDSYRTQMVALDGATPFQSHYQRFTVATFALLDSGSQRALRGLVYLFCSTSACHTSGLETCSTACSTGTTRQRRSSGHRNDTARPQDIVSSPGPVGFEDSYGQEPTLGPTDSNGNSSLRPLLWAVLLLPAVALVLGFGVFVGLSQTWAQKLWESNRQ

SEQ ID NO: 3 - protein, 745 aa, Homo sapiens
MACRQRGGSWSPSGWFNAGWSTYRSISLFFALVTSGNSIDVSQLVNPAFPGTVTCDEREITVEFPSSPGTKKWHASVVDPLGLDMPNCTYILDPEKLTLRATYDNCTRRVHGGHQMTIRVMNNSAALRHGAVMYQFFCPAMQVEETQGLSASTICQKDFMSFSLPRVFSGLADDSKGTKVQMGWSIEVGDGARAKTLTLPEAMKEGFSLLIDNHRMTFHVPFNATGVTHYVQGNSHLYMVSLKLTFISPGQKVIFSSQAICAPDPVTCNATHMTLTIPEFPGKLKSVSFENQNIDVSQLHDNGIDLEATNGMKLHFSKTLLKTKLSEKCLLHQFYLASLKLTFLLRPETVSMVIYPECLCESPVSIVTGELCTQDGFMDVEVYSYQTQPALDLGTLRVGNSSCQPVFEAQSQGLVRFHIPLNGCGTRYKFEDDKVVYENEIHALWTDFPPSKISRDSEFRMTVKCSYSRNDMLLNINVESLTPPVASVKLGPFTLILQSYPDNSYQQPYGENEYPLVRFLRQPIYMEVRVLNRDDPNIKLVLDDCWATSTMDPDSFPQWNVVVDGCAYDLDNYQTTFHPVGSSVTHPDHYQRFDMKAFAFVSEAHVLSSLVYFHCSALICNRLSPDSPLCSVTCPVSSRHRRATGATEAEKMTVSLPGPILLLSDDSSFRGVGSSDLKASGSSGEKSRSETGEEVGSRGAMDTKGHKTAGDVGSKAVAAVAAFAGVVATLGFIYYLYEKRTVSNH

SEQ ID NO: 4 - protein, 424 aa, Homo sapiens
MELSYRLFICLLLWGSTELCYPQPLWLLQGGASHPETSVQPVLVECQEATLMVMVSKDLFGTGKLIRAADLTLGPEACEPLVSMDTEDVVRFEVGLHECGNSMQVTDDALVYSTFLLHDPRPVGNLSIVRTNRAEIPIECRYPRQGNVSSQAILPTWLPFRTTVFSEEKLTFSLRLMEENWNAEKRSPTFHLGDAAHLQAEIHTGSHVPLRLFVDHCVATPTPDQNASPYHTIVDFHGCLVDGLTDASSAFKVPRPGPDTLQFTVDVFHFANDSRNMIYITCHLKVTLAEQDPDELNKACSFSKPSNSWFPVEGSADICQCCNKGDCGTPSHSRRQPHVMSQWSRSASRNRRHVTEEADVTVGPLIFLDRRGDHEVEQWALPSDTSVVLLGVGLAVVVSLTLTAVILVLTRRCRTASHPVSASE

SEQ ID NO: 5 - protein, 350 aa, Homo sapiens
MGPHFTLLCAALAGCLLPAEGCVICDPSVVLALKSLEKDYLPGHLDAKHHKAMMERVENAVKDFQELSLNEDAYMGVVDEATLQKGSWSLLKDLKRITDSDVKGDLFVKELFWMLHLQKETFATYVARFQKEAYCPNKCGVMLQTLIWCKNCKKEVHACRKSYDCGERNVEVPQMEDMILDCELNWHQASEGLTDYSFYRVWGNNTETLVSKGKEATLTKPMVGPEDAGSYRCELGSVNSSPATIINFHVTVLPKMIKEEKPSPNIVTPGEATTESSISLQPLQPEKMLASRLLGLLICGSLALITGLTFAIFRRRKVIDFIKSSLFGLGSGAAEQTQVPKEKATDSRQQ